(19) United States
(12) Patent Application Publication
(10) Pub. No.: US 2024/0335441 A1
(43) Pub. Date: Oct. 10, 2024

(54) METHOD OF TREATING POSTTRAUMATIC STRESS DISORDER

(71) Applicant: Jazz Pharmaceuticals Ireland Limited, Dublin (IE)

(72) Inventor: Kimberly BABSON, Palo Alto, CA (US)

(21) Appl. No.: 18/294,129

(22) PCT Filed: Aug. 3, 2022
(86) PCT No.: PCT/US22/74469

§ 371 Date: Jan. 31, 2024

Related U.S. Application Data
(60) Provisional application No. 63/229,387, filed on Aug. 4, 2021.

(51) Int. Cl. A61K 31/501

(52) U.S. Cl. CPC A61K 31/501

(57) ABSTRACT

This disclosure provides methods for treating a disease or disorder by administering pharmaceutical compounds. In particular, the disclosure relates to treating posttraumatic stress disorder in an individual in need thereof by administering a fatty acid amid hydrolase inhibitor or a pharmaceutically acceptable salt thereof.

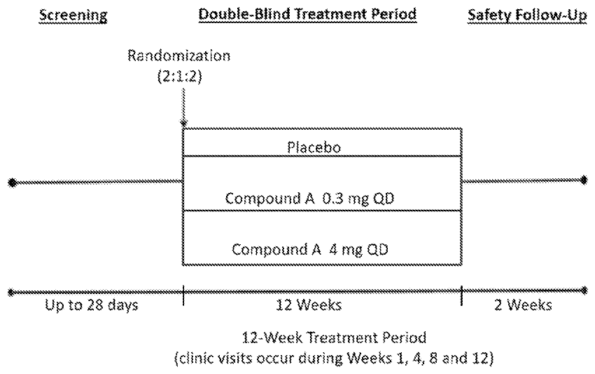

### CROSS-REFERENCE TO RELATED APPLICATION

This application claims priority to and benefit of U.S. Provisional Patent Application No. 63/229,387, filed Aug. 4, 2021, the disclosure of which is hereby incorporated herein by reference in its entirety.

### BACKGROUND

Posttraumatic stress disorder (PTSD) is a common psychiatric condition that can result from direct or indirect exposure to actual or threatened death, serious injury or sexual violence (APA 2013). Approximately 70% of the global population will experience a traumatic event at some point during their lifetime, the majority of whom will recover in the days and weeks following trauma exposure (Kessler 2017). However, 4.7% to 6.1% will go on to develop PTSD in the US (Goldstein 2016), with similar rates observed across European Union countries (0.6 to 6.7%) (Burri and Maercker 2014). While men are more likely to experience a traumatic event, women are twice as likely to develop PTSD (Goldstein 2016; Kessler 2017).

Posttraumatic stress disorder is characterized by symptoms experienced after a potentially traumatic event (Hoffman 2018). Symptoms of PTSD are grouped into 4 symptom clusters: 1) recurrent intrusion/re-experiencing; 2) persistent avoidance; 3) negative alterations in mood and cognition; and 4) marked arousal/reactivity. A traumatic event is defined as exposure to actual or threatened death, serious injury, or sexual violence that is either directly experienced, witnessed, learned about the occurrence to a loved one, or extreme or repeated exposure to aversive details through work (e.g., first responders) (APA 2013). In addition to increased risk for mortality, PTSD also has a significant impact on how patients feel and function. Posttraumatic stress disorder impacts all aspects of patients' lives including negative impacts on work, school, relationships, housing, legal problems, financial problems and an overall increase in healthcare utilization and healthcare costs (Kessler 2017; Watson 2019). Various guidelines exist for the treatment of PTSD (APA 2004; NICE 2018; VA/DoD 2003; ISTSS 2018), most of which include guidance for both psychological and pharmacological treatments. There is an urgent need to address a critical gap in the advancement of pharmacotherapy treatment for PTSD (Krystal 2017).

### SUMMARY

In some aspects, provided here are methods of treating posttraumatic stress disorder. Various embodiments are contemplated herein. In some embodiments, provided is a method of treating posttraumatic stress disorder (PTSD) in an individual in need thereof, comprising administering to the individual an oral dosage form of Compound A:

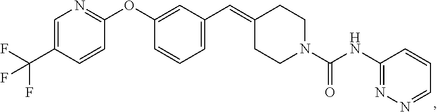 [SMILES: O=C(Nc1cccnn1)N1CCC(=Cc2cccc(Oc3ccc(C(F)(F)F)cn3)c2)CC1]

or a pharmaceutically acceptable salt thereof, once daily (QD) for a period of time of at least 8 weeks.

In some embodiments, the oral dosage form is solid. In certain variations, the oral dosage form comprises at least 0.3 mg of Compound A. In certain variations, the oral dosage form comprises from 0.3 mg to 4 mg of Compound A.

In some embodiments, the period of time is a period of at least 12 weeks. In some variations, the oral dosage form of Compound A is administered to the individual in an amount effective to achieve a plasma concentration of Compound A of between 1 ng/ml and 3 ng/ml. In other variations, the oral dosage form of Compound A is administered to the individual in an amount effective to maintain a plasma concentration of Compound A of at least 1 ng/ml for at least 12 hours. In yet other variations, the oral dosage form of Compound A is administered to the individual in an amount effective to achieve a plasma concentration of endocannabinoid anandamide (AEA) of between 2 ng/ml to 5 ng/ml. In certain variations, the oral dosage form of Compound A is administered to the individual in an amount effective to maintain a plasma concentration of endocannabinoid anandamide (AEA) of at least 2 ng/ml for at least 12 hours.

In some embodiments, the individual in need of treatment is an adult. In one variation, the individual is a human. In certain embodiments, the individual in need of treatment has a genetic predisposition to PTSD.

### DESCRIPTION OF THE DRAWING

The drawing is a schema of a double blind, placebo-controlled, randomized, parallel-group study of the safety and efficacy of Compound A in the treatment of adult participants with PTSD.

### DETAILED DESCRIPTION

### Definitions

As used herein, “treatment” or “treating” refers to an approach for obtaining beneficial or desired results. In the context of PTSD, beneficial or desired results can include reducing or alleviating one or more symptoms associated with posttraumatic stress disorder, defined in the Diagnostic and Statistical Manual of Mental Disorders, Fifth Edition (DSM-5), including but not limited to, recurrent intrusion or re-experiencing, persistent avoidance, negative alterations in mood and cognition, and marked arousal or reactivity, or increasing a patient's quality of life.

As used herein, “pharmaceutically acceptable” refers to a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

As used herein, “an oral dosage form” refers to any formulation suitable for oral administration.

As used herein, “individual” refers to a mammal (e.g., a human) in need of treatment.

The term “adult” refers to an individual 18 years of age or older.

Reference to “about” a value or parameter herein includes (and describes) variations that are directed to that value or parameter per se.

The singular forms “a,” “or,” and “the” include plural referents unless the context clearly dictates otherwise.

The term “comprise” or variations such as “comprises” or “comprising”, will be understood to imply the inclusion of a stated element, integer or step, or group of elements, integers or steps, but not the exclusion of any other element, integer or step, or group of elements, integers or steps.

Embodiments described herein include “consisting” and/or “consisting essentially of” aspects.

This application refers to various issued patent, published patent applications, journal articles, and other publications, each of which is incorporated herein by reference for all purposes.

### Compound A

Compound A, also known as PF-04457845, is an orally available, highly selective and irreversible inhibitor of fatty acid amide hydrolase (FAAH) (Ahn 2011; Johnson 2011; Bonifacio 2020). Compound A is described chemically as N-Pyridazin-3-yl-4-{3-[5-(trifluoromethyl)-2-pyridyloxy]benzylidene}piperidine-1-carboxamide having the structural formula:

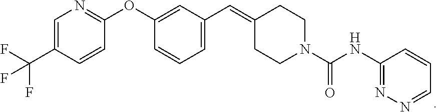 [SMILES: O=C(Nc1cccnn1)N1CCC(=Cc2cccc(Oc3ccc(C(F)(F)F)cn3)c2)CC1]

Compound A is disclosed in U.S. Pat. No. 8,044,052 titled “Biaryl Ether Urea Compounds”, the entirety of which is incorporated herein by reference. Description of Compound A and method of making Compound A can be found in, e.g., Example 5a in column 35 and Example 5b in column 36, of the above referenced patent, which sections are specifically incorporated herein by reference.

A recent investigator-sponsored, placebo-controlled study showed that Compound A enhanced recall of fear extinction memory and blunted negative affect in response to an aversive stimulus in healthy volunteers; see, e.g., Mayo et al. Biological Psychiatry 2020, 87:538-547. Another study revealed that Compound A significantly increased slow wave sleep as measured by polysomnography and improved self-reported overall self-reported sleep compared to placebo in men with cannabis use disorder (CUD) (see, e.g., D'Souza et al. The Lancet Psychiatry 2019, 6:35-45). Compound A has also been undergoing clinical evaluation for the treatment of cannabis withdrawal and cannabis use disorder (see, e.g., NCT 01618656 and NCT 03386487).

### Methods

Methods of treating posttraumatic stress disorder (PTSD) in an individual in need thereof, comprising administering to the individual an oral dosage form of Compound A or a pharmaceutically acceptable salt thereof are provided herein. To achieve treatment, the oral dosage form may be given to the individual for any period of time (e.g., at least or about 4 weeks, 8 weeks, 12 weeks, 4 months, 6 months, 1 year, etc.). The oral dosage form may comprise any amount of Compound A (e.g., at least or about 0.3 mg, 0.5 mg, 1 mg, 2 mg, 3 mg, 4 mg, 5 mg, 6 mg, 7 mg, 8 mg, 9 mg, 10 mg, etc.). Treatment may reduce or alleviate one or more DSM-5-defined symptoms associated with posttraumatic stress disorder, including but not limited to, recurrent intrusion or re-experiencing, persistent avoidance, negative alterations in mood and cognition, and marked arousal or reactivity.

The oral dosage form may be given to the individual for any period of time to achieve treatment. In some embodiments, the period of time is a period of at least 8 weeks. In some embodiments, the period of time is a period of at least 12 weeks. In some embodiments, the period of time is a period of between 1 to 3 months. In some embodiments, the period of time is a period of between 3 to 6 months. In some embodiments, the period of time is a period of 1 year.

In some embodiments, in conjunction with embodiments above or below, the oral dosage form is given to the individual for a period of time of about 4 weeks. In some embodiments, in conjunction with embodiments above or below, the oral dosage form is given to the individual for a period of time of about 2 weeks. In some embodiments, in conjunction with embodiments above or below, the oral dosage form is given to the individual for a period of time of about 1 week.

The oral dosage form may comprise any amount of Compound A or a pharmaceutically acceptable salt thereof to achieve treatment. In some embodiments, the oral dosage form comprises at least or about 0.3 mg of Compound A. In some embodiments, the oral dosage form comprises between about 0.3 mg to about 4 mg of Compound A. In some embodiments, the oral dosage form comprises about any one of between 0.5 mg to 4 mg, or between 1 mg and 4 mg, or between 2 mg and 4 mg, or between 3 mg and 4 mg, or between 2 mg and 3 mg of Compound A. In some embodiments, the oral dosage form comprises at least or about 4 mg (e.g., at least or about 4 mg, 5 mg, 6 mg, 7 mg, 8 mg, 9 mg, 10 mg) of Compound A. In some embodiments, the oral dosage form comprises about any one of between 4 mg to 5 mg, or between 4 mg and 6 mg, or between 4 mg and 7 mg, or between 4 mg and 8 mg, or between 4 mg and 9 mg, or between 4 mg and 10 mg of Compound A.

In some embodiments, the method comprises administering an amount of Compound A effective to achieve a minimum effective concentration (MEC) in blood plasma of Compound A or an active metabolite thereof. In some embodiments, the MEC of Compound A is at least about 1 ng/ml. In some embodiments, the MEC of Compound A is at least or about any one of 1 ng/ml, 1.5 ng/ml, 2 ng/ml, 2.5 ng/ml, 3 ng/ml, 3.5 ng/ml, 4 ng/ml, 4.5 ng/ml, 5 ng/ml, 5.5 ng/ml, 6 ng/ml, 7 ng/ml, 8 ng/ml, 9 ng/ml, or 10 ng/ml. In some embodiments, the MEC of Compound A is between about 1 ng/ml and about 2 ng/ml. In some embodiments, the MEC of Compound A is about any one of 1 ng/ml to 2 ng/ml, 1 ng/ml to 3 ng/ml, 1 ng/ml to 4 ng/ml, 1 ng/ml to 5 ng/ml, 1 ng/ml to 6 ng/ml, 1 ng/ml to 7 ng/ml, 1 ng/ml to 8 ng/ml, 1 ng/ml to 9 ng/ml, 1 ng/ml to 10 ng/ml. In some embodiments, the method comprises administering an amount of Compound A effective to maintain a MEC of Compound A for a duration of time (e.g. at least 6 hours, 12 hours, 15 hours, 18 hours, 24 hours, etc.). For example, in some embodiments, the MEC of Compound A is about any one of 1 ng/ml to 2 ng/ml, 1 ng/ml to 3 ng/ml, 1 ng/ml to 4 ng/ml, 1 ng/ml to 5 ng/ml, 1 ng/ml to 6 ng/ml, 1 ng/ml to 7 ng/ml, 1 ng/ml to 8 ng/ml, 1 ng/ml to 9 ng/ml, 1 ng/ml to 10 ng/ml, maintained for a duration of any one of at least 6 hours, 12 hours, 15 hours, 18 hours, or 24 hours. In some embodiments, administration of Compound A comprises administration of an oral dosage form. In some embodiments, the oral dosage form is administered once daily and achieves the desired MEC for a duration of at least or about 6 hours, 12 hours, 15 hours, 18 hours, or 24 hours.

In some embodiments, the method comprises administering an amount of Compound A effective to achieve a minimum effective concentration (MEC) in blood plasma of endocannabinoid anandamide (AEA). In some embodiments, the MEC of AEA is at least about 2 ng/ml. In some embodiments, the MEC of AEA is at least or about any one of 2 ng/ml, 2.5 ng/ml, 3 ng/ml, 3.5 ng/ml, 4 ng/ml, 4.5 ng/ml, 5 ng/ml, 5.5 ng/ml, 6 ng/ml, 7 ng/ml, 8 ng/ml, 9 ng/ml, or 10 ng/ml. In some embodiments, the MEC of AEA is between about 2 ng/ml and about 3 ng/ml. In some embodiments, the MEC of AEA is about any one of between 2 ng/ml to 3 ng/ml, 2 ng/ml to 4 ng/ml, 2 ng/ml to 5 ng/ml, 2 ng/ml to 6 ng/ml, 2 ng/ml to 7 ng/ml, 2 ng/ml to 8 ng/ml, 2 ng/ml to 9 ng/ml, or 2 ng/ml to 10 ng/ml. In some embodiments, the method comprises administering an amount of Compound A effective to maintain a MEC of AEA for a duration of time (e.g. at least or about 6 hours, 12 hours, 15 hours, 18 hours, 24 hours, etc.). For example, in some embodiments, the MEC of AEA is at least or about any one of 2 ng/ml, 2.5 ng/ml, 3 ng/ml, 3.5 ng/ml, 4 ng/ml, 4.5 ng/ml, 5 ng/ml, 5.5 ng/ml, 6 ng/ml, 7 ng/ml, 8 ng/ml, 9 ng/ml, or 10 ng/ml, maintained for a duration of at least or about any one of 6 hours, 12 hours, 15 hours, 18 hours, or 24 hours.

In some embodiments, the individual in need of treatment is diagnosed with PTSD. For example, PTSD may be diagnosed using the criteria in the Diagnostic and Statistical Manual of Mental Disorders (DSM-5). In some embodiments, the PTSD diagnosis may be confirmed using the Mini International Neuropsychiatric Interview (MINI). The MINI is a short, structured, clinician administered, diagnostic interview for psychiatric disorders (Sheehan 1998). The MINI assessment may be used to confirm PTSD diagnosis. In some embodiments, the PTSD diagnosis may be confirmed by the CAPS-5. In some embodiments, the individual may be diagnosed with PTSD using DSM-5, MINI, CAPS-5, or any combination thereof.

In some embodiments, the individual in need of treatment has a PCL-5 total score of at least 30 (e.g. at least 30, 31, 32, 33, 34, 35, 40, 45, 50, etc.). In some embodiments, the individual in need of treatment has a PCL-5 total score of at least 35. In some embodiments, the individual in need of treatment has a comorbid diagnose of major depressive disorder, persistent depressive disorder, generalized anxiety disorder, specific phobia, obsessive compulsive disorder, social anxiety disorder, or any combination thereof. In some embodiments, the individual in need of treatment is being treated with a selective serotonin reuptake inhibitor (SSRI) or serotonin norepinephrine reuptake inhibitor (SNRI) prior to PTSD treatment using Compound A (e.g., receiving a stable dose of an SSRI or SNRI at least 8 weeks prior to treatment). In some embodiments, PTSD treatment using Compound A is an add-on therapy administered to an individual receiving treatment with an SSRI or SNRI. In some embodiments, PTSD treatment using Compound A is an add-on therapy administered to an individual receiving treatment with an SSRI. In some embodiments, PTSD treatment using Compound A is an add-on therapy administered to an individual receiving treatment with an SNRI. In some embodiments, PTSD treatment using Compound A is a monotherapy administered to an individual. In some embodiments, PTSD treatment using Compound A is administered to an individual who does not concurrently receive a treatment with an SSRI or SNRI.

In some embodiments, the individual in need of treatment has a genetic predisposition to PTSD. In some embodiments, the individual in need of treatment is an adult. In some embodiments, the individual in need of treatment is an adult male. In some embodiments, the individual in need of treatment is an adult female.

### Efficacy Assessments

In some embodiments, treatment may be effective to achieve improvement in one or more of the following evaluations: CAPS-5, CGI-S, CGI-C, PGI-S, PCL-5, B-IPF, ReQOL-10, SDS, PSQI-A, ISI, PHQ-9, and SIGH-A. In some embodiments, treatment may be effective to achieve improvement in CAPS-5, CGI-S, PGI-S, or any combination thereof. For example, treatment may be effective to achieve change in CAPS-5 by at least 5 points (e.g., 5, 6, 7, 8, 9, 10, 15, 20 points, etc.). In some embodiments, treatment may be effective to achieve at least 1 point change in one or more CAPS-5 items (e.g., 1, 2, 3, 4, 5, 6, 7, 8, 9, 10 CAPS-5 items, etc.). In some embodiments, treatment may be effective to achieve at least 30% (e.g., 30%, 35%, 40%, 45%, 50% or more) improvement on the CAPS-5 total symptom severity score. In some embodiments, treatment may be effective to achieve very much or much improved on the CGI-S. In some embodiments, treatment may be effective to achieve at least 1 unit (e.g., 1, 2, 3, 4, 5 units, etc.) of improvement on the PGI-S.

In some embodiments, efficacy of the treatment may be assessed by Clinician Administered PTSD Scale (CAPS-5). The CAPS-5 is a structured, clinician administered, clinical interview wherein participants report on their symptoms of PTSD (Weathers 2018). The CAPS-5 may be administered by qualified medical personnel (e.g., PI, clinical rater). It is a well-established, widely used, and validated assessment that captures PTSD symptom frequency and intensity over the past week using a 5-point Likert-type rating scale. Its distinctive features include standardized prompts, assessment of symptom frequency, intensity, severity and behaviorally anchored ratings. The CAPS-5 total symptom severity score is derived by summing scores for items 1-20. Each item is rated with a single severity score on a five-point rating scale (0-4) corresponding to the following categories: 0 Absent, 1 Mild/subthreshold, 2 Moderate/threshold, 3 Severe/markedly elevated, and 4 Extreme/incapacitating. The CAPS-5 total symptom severity score can range from 0 to 80.

In some embodiments, efficacy of the treatment may be assessed by Clinical Global Impression of Severity (CGI-S). The CGI-S is a 7-point Likert-type rating scale and a widely used assessment in clinical psychopharmacology trials to assess severity of illness (Guy 1976). The responses to this investigator-completed scale range from 1 (Normal, not at all ill) to 7 (Among the most extremely ill patients). The qualified medical personnel rate his/her impression of the severity of the participant's current ability to function due to their PTSD relative to his/her experience with this patient population. In some embodiments, the CGI-S may be used to evaluate the severity of an individual's PTSD.

In some embodiments, efficacy of the treatment may be assessed by Clinical Global Impression of Change (CGI-C). The CGI-C is a 7-point Likert-type rating scale and a widely used assessment to assess efficacy in clinical drug trials (Guy 1976). Investigators or trained raters rate their impression of any change in the severity of the individual's condition since Baseline on a 7-point scale ranging from 1 (very much improved) to 7 (very much worse). Ratings focus on the individual's change in their ability to function due to PTSD.

In some embodiments, efficacy of the treatment may be assessed by Patient Global Impression of Severity (PGI-S). The PGI-S is a 5-point Likert-type rating scale and a widely used assessment in clinical psychopharmacology trials to assess severity of illness. The responses of this patient-completed scale range from 1=none to 5=very severe. Individuals may report on the severity of their PTSD symptoms.

In some embodiments, efficacy of the treatment may be assessed by Brief Inventory of Psychosocial Functioning (B-IPF). The B-IPF is a 7-item self-reported questionnaire that assesses PTSD-related psychosocial functional impairment (Kleiman 2018). Questions regarding functional impairment are rated on a 6-point scale from 0 (Not at all), 1 to 5 (Somewhat), to 6 (Very much).

In some embodiments, efficacy of the treatment may be assessed by Sheehan Disability Scale (SDS). The SDS is a brief, 5-item self-reported tool that assesses functional impairment in work/school, social life, and family life (Leon 1997). The total score is 0 to 30 (0 unimpaired, 30 highly impaired). High scores are associated with significant functional impairment.

In some embodiments, efficacy of the treatment may be assessed by PTSD Checklist (PCL-5). The PCL-5 is a 20-item self-reported measure that assesses the 20 DSM-5 symptoms of PTSD. It can be used for monitoring symptom change during and after treatment (Blevins 2015).

In some embodiments, efficacy of the treatment may be assessed by Insomnia Severity Index (ISI). The ISI is a 7-item self-reported questionnaire assessing the nature, severity, and impact of insomnia (Morin 2011). The dimensions evaluated are: severity of sleep onset, sleep maintenance, and early morning awakening problems, sleep dissatisfaction, interference of sleep difficulties with daytime functioning, noticeability of sleep problems by others, and distress caused by the sleep difficulties. A 5-point Likert scale is used to rate each item.

In some embodiments, efficacy of the treatment may be assessed by Pittsburgh Sleep Quality Index-PTSD Addendum (PSQI-A). The PSQI-A is a 19-item self-report questionnaire evaluating sleep quality and disturbances over the past month (Insana 2013). The PSQI-A is comprised of seven different sleep disturbance items that are commonly reported by adults with PTSD (eg, hot flashes, memories or nightmares of the traumatic experience, and episodes of terror during sleep). Items are rated on a 0 (not in the past month) to 3 (three or more times a week) point scale, and can be summed to create a total score. The total scores can range from 0 (normal) to 21 (severe).

In some embodiments, efficacy of the treatment may be assessed by Patient Health Questionnaire (PHQ-9). The PHQ-9 is a component of the longer Patient Health Questionnaire and is a concise, self-administered tool for assessing depression (Kroenke 2001). It incorporates DSM depression criteria with other leading major depressive symptoms into a brief self-report instrument that is commonly used for screening and diagnosis, as well as selecting and monitoring treatment.

In some embodiments, efficacy of the treatment may be assessed by Structured Interview Guide for the Hamilton Anxiety Rating Scale (SIGH-A). The SIGH-A is a clinician administered rating scale developed to measure the severity of anxiety symptoms (Hamilton 1959). The scale consists of 14 items, each defined by a series of symptoms, and measures both psychic anxiety (mental agitation and psychological distress) and somatic anxiety (physical complaints related to anxiety).

In some embodiments, efficacy of the treatment may be assessed by Recovering Quality of Life (ReQOL). The ReQoL-10 is a patient reported outcome that has been developed to assess the quality of life for people with different mental health conditions (Keetharuth 2018). It is suitable for use across all mental health populations including common mental health problems.

### Administration

In some embodiments, the oral dosage form may be solid (e.g., tablet or capsule). In some embodiments, the oral dosage form may be liquid. In some embodiments, the oral dosage form is administered to the individual once daily (QD). In some embodiments, the oral dosage form is administered to the individual in the morning (e.g., any time from about 6 am to about noon). In some embodiments, the oral dosage form is administered to the individual within about 4 hours (e.g., within about 4, 3, 2, or 1 hour) after waking. In some embodiments, the oral dosage form is administered to the individual in the morning QD without regard for food (e.g., with or without food).

Other administration routes may also be used, which include but are not limited to: parenteral (e.g., intravenous, subcutaneous, intramuscular, intraperitoneal, or intrapleural) and transdermal administration routes.

### EXAMPLES

### Example 1. A phase 2 study to assess the safety and efficacy of Compound A in the treatment of adults with PTSD

### Study Design

This study is a 12-week, double blind, placebo-controlled, randomized, parallel-group study of the safety and efficacy of Compound A in the treatment of adult participants with PTSD. The purpose of this phase 2 study is to provide an evaluation of the efficacy and safety of Compound A in participants with PTSD, while evaluating a broad range of Compound A doses to support pharmacokinetic (PK)/pharmacodynamics (PD) exposure-response analysis.

Participants who meet Diagnostic and Statistical Manual of Mental Disorders, 5th Edition (DSM-5) criteria for PTSD as determined by the Mini International Neuropsychiatric Interview (MINI), with a total PCL-5 score ≥35 at Screening and Baseline will be eligible for enrollment. The PTSD diagnosis must be confirmed by the CAPS-5 administered at Baseline. Following screening, eligible participants (approximately 270 participants) will be randomized (2:1:2) to 1 of 3 treatment arms: placebo, Compound A 0.3 mg QD, or Compound A 4 mg QD. Randomization will be stratified based on presence versus absence of use of a stable selective SSRIs/serotonin norepinephrine reuptake inhibitors (SNRIs) at Screening. The sponsor, study investigators, clinical raters, and participants will be blinded to treatment assignment.

The study will consist of the following periods: screening period and washout of prohibited medications (up to 28 days), baseline and randomization (Day 1), double-blind treatment period (12 weeks, e.g., Week 1 to Week 12), and safety follow-up (2 weeks, e.g., 14 days after last dose). During the 12-week treatment periods, clinical visits occur during Weeks 1, 4, 8 and 12. A study schema is shown in the drawing.

### Study Population

Participants are eligible to be included in the study only if all of the following criteria apply: 1. Participant must be 18 to 70 years of age inclusive, at the time of signing the informed consent. 2. Participants must be outpatients with a primary diagnosis of DSM-5 defined PTSD based on the MINI at Screening and confirmed by the CAPS-5 at Baseline. 3. PCL-5 total score ≥35 at Screening and Baseline. 4. PTSD must be the primary diagnosis. Participants with comorbid diagnoses of major depressive disorder (with the exception of current severe symptoms), persistent depressive disorder (previously known as dysthymia), generalized anxiety disorder, specific phobia, obsessive compulsive disorder (with the exception of severe symptoms), or social anxiety disorder, are eligible if PTSD is the primary diagnosis. 5. Participants prescribed an SSRI or SNRI must be on a stable dose for at least 8 weeks prior to screening with no plan to change dose or treatment regimen during the course of the trial. Those not on an SSRI/SNRI at screening may not start treatment with a SSRI/SNRI during the course of the study.

### Study Objectives and Assessments

The primary objective of this study is to evaluate the efficacy of Compound A administered once daily for up to 12 weeks at doses of 0.3 mg and 4 mg compared to placebo in the treatment of adults with PTSD, as measured by the change in CAPS-5 total symptom severity score. The variable (endpoint) for the primary estimand is Clinician Administered PTSD Scale (CAPS-5) total symptom severity score change from Baseline to Week 12.

The key secondary objective of this study is to evaluate the efficacy of Compound A administered once daily for 12 weeks at doses of 0.3 mg and 4 mg compared to placebo on overall clinician and patient global impression of severity. The endpoints for the key secondary estimand include 1) change in Clinical Global Impression of Severity (CGI-S) from Baseline to Week 12 and 2) change in Patient Global Impression of Severity (PGI-S) from Baseline to Week 12.

Secondary objectives of this study include evaluating the efficacy of Compound A administered once daily for 12 weeks at doses of 0.3 mg and 4 mg compared to placebo on functional outcomes and patient reported symptoms in adults with PTSD. The variables (endpoints) for the estimands are: 1) Responder analysis which defined in 3 separate ways: i) Percent of participants with ≥30% improvement on the CAPS-5 total symptom severity score from Baseline to Week 12; ii) Percent of participants who are very much or much improved on the CGI-C at Weeks 4 and 12; iii) Percent of participants with ≥1 unit of improvement on the PGI-S from Baseline to Week 12. 2) CAPS-5 total symptom severity score change from Baseline to Week 4. 3) Change in functional outcomes measured by the Brief Inventory of Psychosocial Functioning (B-IPF) and Sheehan Disability Scale (SDS) from Baseline to Week 12. 4) Change in patient and clinician-reported symptoms of PTSD as assessed by the PTSD Checklist (PCL-5), PGI-S, and CGI-S from Baseline to Weeks 1, 4, 8, and 12. 5) Change in self-reported sleep problems as measured by the Insomnia Severity Index (ISI) and Pittsburgh Sleep Quality Index-PTSD Addendum (PSQI-A) from Baseline to Week 12. 6) Change in PTSD symptom clusters (re-experiencing, avoidance, arousal, and mood) as assessed by the change in the sub-scales of the CAPS-5 from Baseline to Week 12. 7) Change in mood and anxiety from Baseline to Weeks 4, 8 and 12 as assessed by the Patient Health Questionnaire (PHQ-9) and the Structured Interview Guide for the Hamilton Anxiety Scale (SIGH-A). 8) Change in quality of life from Baseline to Week 12 as measured by the Recovering Quality of Life (ReQoL).

Secondary objectives of this study also include evaluating the efficacy of Compound A administered once daily for 12 weeks at doses of 0.3 mg and 4 mg compared to placebo on functional outcomes and patient reported symptoms in adults with PTSD. The safety of Compound A will be evaluated by the following assessments: AEs, vital signs, physical examination, 12-lead ECG, clinical laboratory tests (chemistry, hematology, urinalysis), Columbia Suicide Severity Rating Scale (C-SSRS), and withdrawal scale (marijuana withdrawal checklist).

Secondary objectives of this study further include characterizing the PK and PD of Compound A in adults with PTSD using population PK modeling and simulation methodology, which will be assessed by plasma concentration of AEA, Compound A and its metabolites.

Exploratory objective is to assess the association between natural genetic variation in select genes on clinical endpoints and/or the incidence of AEs. A blood sample will be collected at baseline for genotyping single nucleotide polymorphisms (SNPs) relevant to Compound A pharmacology (e.g., FAAH and CNR1) as well as exploratory SNPs linked to the clinical condition under study. Pharmacogenomics analysis via Whole Exome Sequencing may be performed at the end of the study.

### Pharmacokinetics

Blood samples for measurement of plasma concentration of Compound A and its metabolites will be collected on Week 1, Week 8, and Week 12. Blood samples of 4 mL will be drawn at predose and 1 sample at 2 (+15 minutes) hours post dose on Week 1 and Week 12, 5 (+15 minutes) and 8 (+15 minutes) hours post dose on Week 8. Week 12 post-dose sample should be collected after completion of the CAPS-5 assessment. Serial blood samples will be taken within 5 minutes of the post-dose nominal time.

### Pharmacogenomics

A 10 mL blood sample for DNA isolation will be collected from participants who have consented to participate in the genetic analysis component of the study on Day 1 (e.g., baseline and randomization).

### Biomarkers

Blood samples of approximately 4 mL will be collected to examine relevant PD biomarkers (e.g., plasma AEA concentration, FAAH activity) on Day 1, Week 1, Week 8, Week 12, and 14 days after last dose.

### Other Embodiments

The foregoing has been described of certain non-limiting embodiments of the present disclosure. Those of ordinary skill in the art will appreciate that various changes and modifications to this description may be made without departing from the spirit or scope of the present disclosure, as defined in the following claims.

1. A method of treating posttraumatic stress disorder (PTSD) in an an individual in need thereof, comprising administering to the individual an oral dosage form comprising from about 0.3 mg to about 4 mg of a compound having the structure: 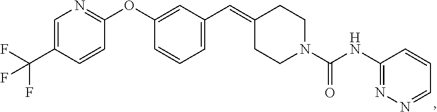 [SMILES: O=C(Nc1cccnn1)N1CCC(=Cc2cccc(Oc3ccc(C(F)(F)F)cn3)c2)CC1]
or a pharmaceutically acceptable salt thereof, once daily (QD) for a period of time of at least 8 weeks.

2. (canceled)

3. The method of claim 1, wherein the oral dosage form comprises about 0.3 mg of the compound, or a pharmaceutically acceptable salt thereof.

4. The method of claim 1, wherein the oral dosage form comprises about 4 mg of the compound, or a pharmaceutically acceptable salt thereof.

5. The method of claim 1, wherein the period of time is a period of at least 12 weeks.

6. The method of claim 1, wherein the oral dosage form of the compound, or a pharmaceutically acceptable salt thereof, is administered to the individual in an amount effective to achieve a plasma concentration of the compound, or a pharmaceutically acceptable salt thereof, of from about 1 ng/ml to about 3 ng/ml.

7. The method of claim 1, wherein the oral dosage form of the compound, or a pharmaceutically acceptable salt thereof, is administered to the individual in an amount effective to maintain a plasma concentration of the compound, or a pharmaceutically acceptable salt thereof, of at least about 1 ng/ml for at least 12 hours.

8. The method of claim 1, wherein the oral dosage form of the compound, or a pharmaceutically acceptable salt thereof, is administered to the individual in an amount effective to achieve a plasma concentration of endocannabinoid anandamide (AEA) of from about 2 ng/ml to about 5 ng/ml.

9. The method of claim 1, wherein the oral dosage form of the compound, or a pharmaceutically acceptable salt thereof, is administered to the individual in an amount effective to maintain a plasma concentration of endocannabinoid anandamide (AEA) of at least about 2 ng/ml for at least 12 hours.

10. (canceled)

11. The method of claim 1, wherein the individual in need of treatment has a genetic predisposition to PTSD.

12. The method of claim 1, wherein the individual in need of treatment does not have a comorbid diagnosis of major depressive disorder.

13. The method of claim 1, wherein the individual in need of treatment does not concurrently receive a treatment with a selective serotonin reuptake inhibitor (SSRI) or a serotonin norepinephrine reuptake inhibitor (SNRI).

14. The method of claim 1, wherein the individual in need of treatment is not being treated with a selective serotonin reuptake inhibitor (SSRI) or serotonin norepinephrine reuptake inhibitor (SNRI) prior to administering the oral dosage form of the compound, or a pharmaceutically acceptable salt thereof.

15. The method of claim 1, wherein the oral dosage form is administered to the individual as a monotherapy.

16. The method of claim 1, wherein the individual in need of treatment is an adult.

17. The method of claim 1, wherein the individual in need of treatment is an adult male.
